(19) United States
(12) Patent Application Publication
(10) Pub. No.: US 2022/0071538 A1
(43) Pub. Date: Mar. 10, 2022

(54) SYSTEMS AND METHODS FOR COLLECTING BIOMETRIC INFORMATION

(71) Applicant: OpenBCI, Inc., Brooklyn, NY (US)

(72) Inventors: Conor Russomanno, Brooklyn, NY (US); Aaron Trocola, Brooklyn, NY (US); Sean Montgomery, Reno, NV (US); Shirley ZHANG, Brooklyn, NY (US); Joseph ARTUSO, Brooklyn, NY (US); Eva ESTEBAN, Brooklyn, NY (US)

(73) Assignee: OpenBCI, Inc., Brooklyn, NY (US)

(21) Appl. No.: 17/528,635

(22) Filed: Nov. 17, 2021

Related U.S. Application Data
(63) Continuation-in-part of application No. PCT/US2021/015470, filed on Jan. 28, 2020.
(60) Provisional application No. 63/114,792, filed on Nov. 17, 2020.

(51) Int. Cl. A61B 5/256; A61B 5/021; A61B 5/024; A61B 5/291; A61B 5/296; A61B 5/297; A61B 5/00

(52) U.S. Cl. CPC A61B 5/256; A61B 5/02141; A61B 5/02427; A61B 5/291; A61B 5/296; A61B 2562/18; A61B 5/0002; A61B 2562/0247; A61B 2562/166; A61B 2562/164; A61B 5/297

(57) ABSTRACT

Biometric information about a person may be collected and analyzed to gain insight into the person's physical and/or emotional conditions. The collection and analysis may be performed using a uniquely designed sensing device that includes multiple sets of sensors configured to collect EEG, EOG, EMG, EDA, and/or PPG signals from the person's head and/or facial areas. The sensing device may include a multi-layered facepad and may be coupled to a VR/AR headset and/or a scalp engagement apparatus to monitor the person's physiological and/or neural reactions to audio/visual stimuli.

### CROSS REFERENCE TO RELATED APPLICATIONS

This application claims the benefit of Provisional U.S. Patent Application No. 63/114,792 filed Nov. 17, 2020, and is a continuation-in-part of PCT/US2021/015470 filed Jan. 28, 2021 which claims the benefit of priority from Provisional U.S. Patent Application No. 63/114,792 filed Nov. 17, 2020. The above-mentioned applications are incorporated herein by reference in their entireties.

### BACKGROUND

Biometric information about a person may be used to gain insight into the person's physiological and emotional state or conditions. One way of collecting the biometric information is to deliver a stimulus to the person to evoke a sensory or behavioral response and measure the response using one or more sensors. Traditional forms of stimuli used to accomplish this purpose are generally unnatural and oversimplified. As a result, augmented reality (AR) and virtual reality (VR) based technologies have been increasingly used in recent years as means to deliver realistic stimuli to a subject and elicit natural physiological and neural reactions from the subject. While these AR/VR-based technologies have opened new avenues for scientific research and consumer entertainment, there is a lack of biometrics collection systems or devices that can fully release the potential of the technologies. For example, presently available systems and devices are capable of collecting only a specific type of information from a particular area of the human body. These systems and devices are also built with components that are prone to wear and tear, expensive and/or difficult to replace, and uncomfortable for a subject to wear.

Accordingly, it is highly desirable for biometrics sensing and collection apparatus to be capable of collecting and synchronizing multiple types of biometric information, and achieving these objectives using comfortable, embeddable, and/or replaceable components. This way not only will the application range of the collected biometric information be increased, the usability and comfort of the apparatus will also be improved.

### SUMMARY

Described herein are systems, methods and instrumentalities associated with collecting and processing biometric signals from a user. A device comprising a multi-layered facepad may be used to sense the biometric signals. The multi-layered facepad may comprise a first layer comprising a plurality of openings and multiple sublayers, a second layer comprising a circuit board, a third layer comprising a compressible material configured to provide electromagnetic shielding for the second layer, and a fourth layer (e.g., a gasket) configured to secure the first layer, the second layer, and the third layer to the device. The second layer may be configured to be sandwiched between the first layer and the third layer, and the third layer may be configured to be sandwiched between the second layer and the fourth layer. The multiple sublayers of the first layer may include a surface finish sublayer configured to contact a user's face, an ethylene vinyl acetate (EVA) sublayer capable of being molded into different shapes, a memory foam sublayer, and/or an electromagnetic shielding sublayer configured to provide electromagnetic shielding for the circuit board.

The circuit board of the second layer may be a flexible circuit board capable of deformation when pressure is applied to the circuit board or when the circuit board is bent or curved to fit a user's face. The circuit board may include a plurality of sensors, at least one of which may be configured to pass through corresponding at least one of the plurality of openings to detect one or more of the biometric signals from the user that may indicate electroencephalography (EEG) information, electrooculography (EOG) information, electromyography (EMG) information, and/or electrodermal activity (EDA) information about the user. The device may further comprise a photoplethysmography (PPG) circuit board (e.g., a PPG PCB) configured to obtain PPG information about the user. The PPG PCB may be configured to be coupled to the circuit board of the second layer and transmit the PPG information about the user to the circuit board of the second layer. The PPG PCB may include a plurality of optical sensors configured to sense optical signals that are indicative of the PPG information about the user.

The sensors described herein may each include an electrode configured to be secured to the circuit board via a female snap connector. The electrode may include a contact surface made of a conductive material (e.g., such as a metal) and configured to contact a user's face when the device is secured to the user's face. The electrode may comprise a base that forms a part of a conductive path for a signal sensed via the contact surface. Part of the electrode may be shaped as a male connector capable of being snapped into and out of the female snap connector.

The facepad described herein may be used in conjunction with a scalp engaging device that includes a top side, a bottom side opposite the top side, a first printed circuit board (PCB) mounting receptacle on the top side, a plurality of electrode mounting receptacles on the bottom side, an extendable midline rail coupled to and running between the top side and the bottom side of the scalp engagement device, and a plurality of electrodes each configured to be removably hosted in a respective one of the electrode mounting receptacles of the scalp engagement device. The plurality of electrodes may be configured to contact the user's scalp and collect biometric signals therefrom.

The electrodes of the scalp engagement device may each include a sabot assembly, a circuit board, and a conductive contact assembly. The sabot assembly may be configured to be removably coupled with the respective one of the electrode mounting receptacles, and may comprise a spring configured to provide pressure relief to the user's scalp, a cap configured to operate as a backstop of the spring, and a casing coupled with the cap and configured to host the spring. The casing may include a post configured to hold the spring in place, a protrusion configured to fit into a locking track of the cap, and a channel configured to allow wiring to pass from the circuit board to outside the electrode. The electrode may be made modular, allowing for one or more of the sabot assembly, the circuit board, or the conductive contact assembly to be replaced.

The conductive contact assembly of each of the electrodes may comprise an array of flexible prongs arranged in a concentric pattern. Each of these flexible prongs may have a substantially oblique conical shape with an apex of the conical shape arranged radially away from a base of the conical shape, making the prongs capable of extending through the user's hair and contacting the user's scalp. The flexible prongs may be made of a conductive polymer and may be configured to flex outwardly from a center point to intersect the user's scalp when downward force is applied upon the electrode. The conductive contact assembly may further comprise a substantially convex bed with a raised center that tapers off toward a perimeter of the bed and wherein application of downward force upon each of the electrodes as it engages the user's scalp causes the flexible prongs intersecting the user's scalp to flex radially outwardly from the center point and further causes the perimeter of the bed to flex toward an electrically conductive surface of the circuit board.

### BRIEF DESCRIPTION OF THE DRAWINGS

A more detailed understanding of the examples disclosed herein may be had from the following description, given by way of example in conjunction with the accompanying drawing.

FIG. 1 is a diagram illustrating an example system for collecting biometric information about a user.

FIGS. 2A-2D are diagrams illustrating an example sensing device configured to collect biometric signals from a user.

FIGS. 3A and 3B are diagrams illustrating examples of the sensing device described herein.

FIGS. 4A-4C are diagrams illustrating an example front layer of the sensing device described herein.

FIGS. 5A-5D are diagrams illustrating an example printed circuit board (PCB) layer of the sensing device described herein.

FIG. 6 is a diagram illustrating example locations from which biometric signals may be collected by the sensing device described herein.

FIG. 7 is a diagram illustrating an example of a photoplethysmography (PPG) PCB.

FIG. 8 is a diagram illustrating an example electrode that may be included in the sensing device described herein.

FIG. 9A is a diagram illustrating an example of a scalp engagement device configured to collect biometric signals from a user.

FIGS. 9B and 9C are diagrams illustrating a guide arm and a ribbon cable guide that may be included in the scalp engagement device of FIG. 9A.

FIGS. 9D and 9E are diagrams illustrating one or more PCBs that may be included in the scalp engagement device of FIG. 9A.

FIGS. 9F-9I are diagrams illustrating electrodes that may be included in the scalp engagement device of FIG. 9A.

FIGS. 9J and 9K are diagrams illustrating an adjustment mechanism for the face sensing device of FIG. 3A and 3B and the connection to the guide rail of FIG. 9A.

FIG. 10 is a diagram illustrating example locations from which biometric signals may be collected by the scalp engagement device of FIG. 9A.

FIG. 11A is a diagram illustrating an example of an electrode that may be included in the scalp engagement device of FIG. 9A.

FIGS. 11B and 11C are diagrams illustrating an example conductive contact assembly of the electrode shown in FIG. 11A.

FIGS. 11D and 11E are diagrams illustrating an example wire strain relief guide for the electrode shown in FIG. 11A.

FIGS. 12A-12C are diagrams illustrating examples of conductive prongs that may be included in the electrode shown in FIG. 11A.

FIGS. 13A and 13B are diagrams illustrating example padding for the electrode shown in FIG. 11A.

### DETAILED DESCRIPTION

The present disclosure is illustrated by way of example, and not by way of limitation, in the figures of the accompanying drawings.

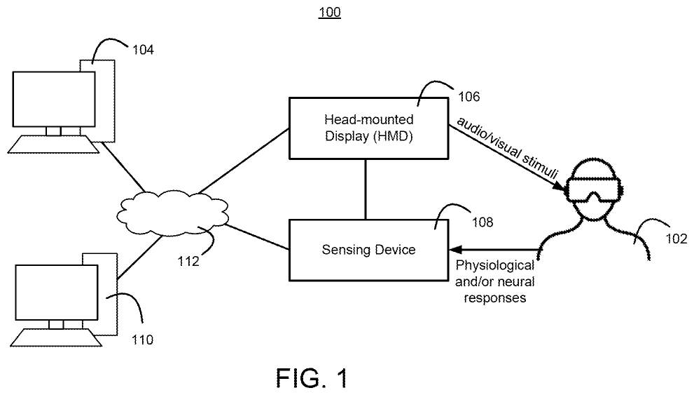

FIG. 1 is a diagram illustrating an example system 100 for collecting and/or analyzing biometric information about a user 102. Such biometric information may include, for example, electroencephalogram (EEG) information, electrooculography (EOG) information, electrodermal activity (EDA) information, photoplethysmography (PPG) information, and/or electromyography (EMG) information about the user that indicate the user's physiological and/or neural reactions to audio and/or visual stimuli. The audio and/or visual stimuli may be generated, for example, based on AR/VR contents provided by a content source (e.g., a server 104) and delivered to the user via a head-mounted device such as a head-mounted display (HMD) 106 (e.g., a VR or AR headset). The HMD 106 may be electrically and/or communicatively coupled to a sensing device 108 (e.g., a head-mounted sensing device) configured to sense and/or collect biometric signals from the user 102 that may be used to generate the aforementioned information. The sensing device 108 may be communicatively coupled to the server 104 and/or one or more other external devices such as one or more additional computing devices 110 and exchange information with these server(s)/device(s) via a communication link 112 (e.g., a wired or wireless communication link). For example, the sensing device 108 may be configured to receive control information (e.g., operating parameters or settings for one or more components of the sensing device) from one or more of the server(s)/device(s) and transmit (e.g., report) the biometric information collected by the sensing device (e.g., raw biometric data and/or analytics generated therefrom) to these server(s)/device(s). As another example, the sensing device 108 may be configured to receive (e.g., extract) timing information (e.g., from the HMD 106, the server 104, or the computing device(s) 110) for the AR/VR contents and link the physiological/neural reactions of the user 102 (e.g., as indicated by the collected biometric information) to respective parts of the AR/VR content based on the timing information.

The biometric information described herein may be used (e.g., by the server 104 and/or the computing device(s) 110) for various purposes including, for example, to evaluate the physical and/or emotional state or conditions of the user 102 in response to the AR/VR contents, to adapt the AR/VR contents being delivered to the user 102 and/or create new contents for the user 102 based on the user's reactions, to enhance the AR/VR experiences of the user 102 by providing feedback to the user and allowing the user to improve his or her skills (e.g., gaming skills) in the immersive environment based on the feedback, to control a device (e.g., a computer or other digital/electronic device) based on a physiological indication by the user (e.g., eye blinks of the user may be used as an indication to initiate a click on a computer), to conduct scientific or commercial research that may require simultaneous collection of multiple types of biometric data, etc.

The sensing device 108 may be configured and/or calibrated, for example, during installation (e.g., setup) of the device and/or while the device is carrying out normal operations (e.g., subsequent to post installation). For instance, the sensing device 108 may (e.g., automatically) detect and/or establish connection to one or more external devices such as the HMD 106, the server 104, and/or the computing device(s) 110 during configuration and/or calibration of the sensing device, or a user of the sensing device 108 may (e.g., manually) connect the sensing device 108 to the aforementioned external devices during the configuration and/or calibration of the sensing device. The sensing device 108 may receive control information from the one or more external devices and configure components (e.g., biometric sensors) of the sensing device based on the control information. Such control information may include, for example, operating parameters of the sensing device 108 such as the types of information to be collected and/or the locations from which to collect the information. The control information may also indicate a destination (e.g., the server 104, the computing device(s) 110, a 3D engine associated with the HMD 106, a cloud service, etc.) to which to the collected biometric information is to be transmitted, e.g., via a communication circuit and/or an application programming interface (API). The API may allow a third party program (e.g., a program written with common programming languages such Python, C++, Java, Julia, and/or scientific protocols such as Lab Streaming Layer) to access the biometric information collected by the sensing device 108, for example, if the third party program has been authorized and/or authenticated to access the biometric information. The authorization and/or authentication may be established based on security rules and/or policies configured for the sensing device 108, for example, during the installation process described herein and/or using the control information described herein. The biometric information transmitted by the sensing device 108 and/or retrieved from the sensing device 108 may be stored and/or processed (e.g., by the receiving device) in real time (e.g., as the biometric information is being collected).

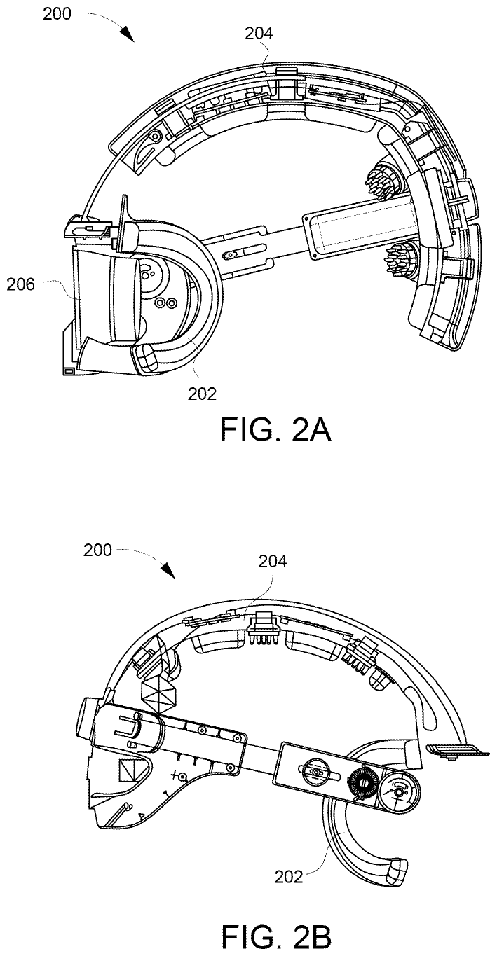

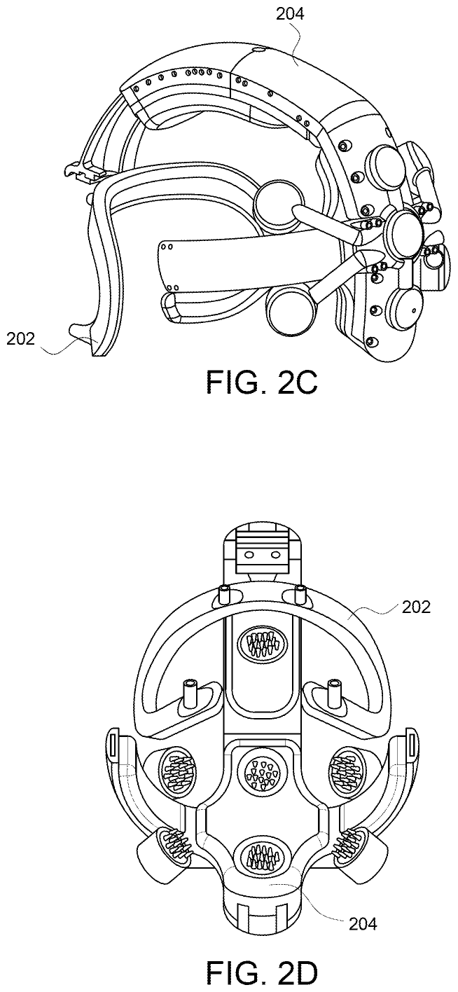

FIGS. 2A-2D show a head-mounted apparatus 200 that includes an example 202 of the sensing device described herein (e.g., the sensing device 108 of FIG. 1). It should be noted that while FIGS. 2A-2D (and FIG. 1) depict the sensing device as being used in conjunction with other head-mounted devices (e.g., such as the HMD 106), the sensing device may also be deployed without the other head-mounted devices, for example, in a standalone setting (e.g., the sensing device may collect biometric information from a user in a non-VR/AR setting). So, the examples shown in FIG. 1 and FIGS. 2A-2D should not be interpreted as requiring that the sensing device be used only in an AR/VR environment and/or with an AR/VR headset.

As shown in FIGS. 2A-2D, the sensing device 202 may be coupled to a mounting device 204 configured to secure (e.g., strap) the head-mounted apparatus 200 to a user's head. The sensing device 202 and/or the mounting device 204 may be additionally coupled to a display device 206 configured to deliver audio/visual stimuli to the user to evoke physiological and/or neural reactions from the user. The head-mounted apparatus 200 may include custom connectors (not shown) for coupling the sensing device 202, the mounting device 204, and/or the display device 206 together. For example, the sensing device 202 may be configured to be coupled to the mounting device 204 and/or the display device 206 via one or more snap connectors so that when the head-mounted apparatus is secured to the user's head, the sensing device 202 may contact one or more areas of the user's face (e.g., the forehead and/or areas surrounding the user's eyes) from where biometric signals may be collected to determine the user's physiological and/or neural reactions to the audio/visual stimuli. These biometric signals may be of different types including, for example, EEG signals, EOG signals, EDA signals, PPG signals, and/or EMG signals that may respectively indicate changes in the user's brain, eyes, skin, heart, and/or muscles in response to the audio/visual stimuli.

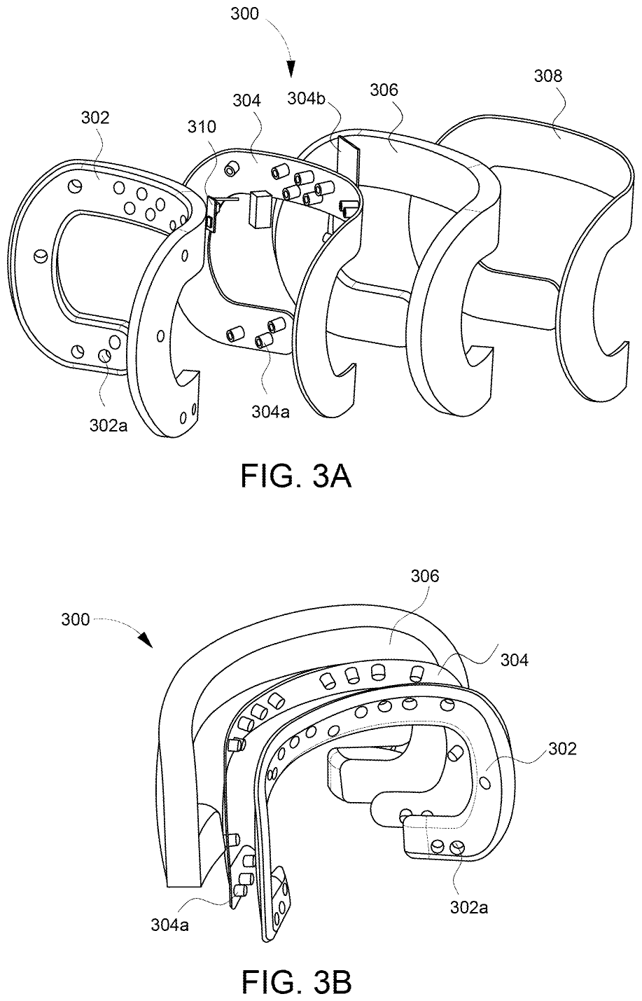

FIGS. 3A and 3B show examples 300 of the sensing device described herein (e.g., the sensing device 108 in FIG. 1 and/or 202 in FIGS. 2A-2D). As shown, the sensing device 300 may include a facepad that comprises multiple layers. For instance, the sensing device 300 may include a first layer 302, a second layer 304, and a third layer 306. The first layer 302 (e.g., which may also be referred to herein as a front layer or front pad) may be configured to contact a user's face and may include multiple openings 302a through which a plurality of sensors may pass to collect signals from the user's face. The second layer 304 (e.g., which may also be referred to herein as a PCB layer) may include a circuit board configured to be installed between the first layer 302 and a third layer 306. The circuit board may be flexible (e.g., deformable under pressure) and may include a plurality of sensors 304a (e.g., electrodes) and/or circuitry 304b (e.g., a flexible PCB). The sensors 304a may be configured to pass through corresponding openings 302a of the first layer 302 to contact the user's face and collect biometric signals from the user's face. The sensors 304a may be electrically coupled to the circuitry 304b, which may be configured to receive the signals sensed/detected by the sensors 304a and process (e.g., pre-process) the signals to fulfill the various purposes described herein. The third layer 306 of the sensing device 300 may be configured to provide electromagnetic field (EMF) shielding for one or more electrical components (e.g., the flexible PCB) of the sensing device, so that those electrical components may be insulated from the electrical noise in the environment as well as the electrical noise generated by a device connected to the facepad (e.g., such as an attached HMD). The third layer 306 may be made from a compressible material (e.g., memory foam) to provide pressure relief to the user's face and/or components of the sensing device 300.

In examples, the sensing device 300 may further include a fourth layers 308 (e.g., a gasket) configured to secure the first layer 302, the second layer 304, and the third layer 306 to the sensing device and/or to connect the sensing device to other devices. In these examples, the third layer 306 may be installed between the PCB layer 304 and the fourth layer 308, and serve as a cushion for the sensors 304a and/or circuitry 304b of the PCB layer 304. The third layer 306 may also operate as a spring behind the sensors 304a to increase the comfort level of the sensing device 300 to the user's face (e.g., by reducing the pressure exerted by the sensors 304a on the user's face). Since the PCB layer 304 is flexible (e.g., a flexible PCB), it may be embedded between the third layer 306 and the fourth layer 308, and adapt its shape to accommodate the pressure caused by the first layer 302 pressing against the user's face and/or the fourth layer 308 flexing to accommodate the curvature of the user's face.

As will be described in greater detail below, including multiple layers of padding in the sensing device 300 and nesting the electronics of the sensing device within these layers may serve to alleviate the pressure a user may feel when using the sensing device (e.g., by distributing the pressure across multiple areas of the user's face). The layers surrounding the electrical components of the sensing device may also protect those components from wear and tear. And since the layers may be individually replaceable, they will also reduce the costs associated with maintaining (e.g., replacing parts of) the sensing device.

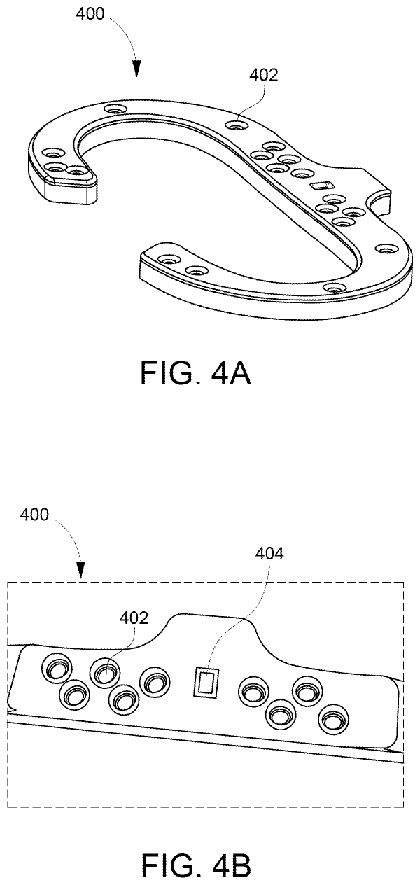

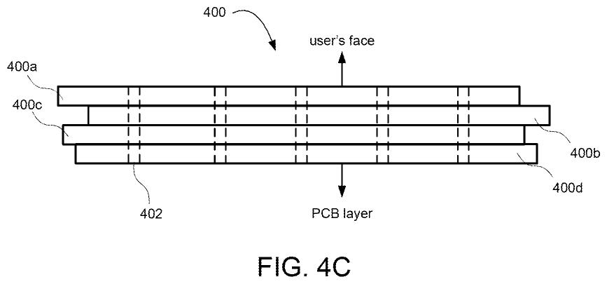

FIGS. 4A-4C show an example front layer 400 (e.g., the first layer 302 in FIGS. 3A and 3B) of the sensing device described herein. As shown in FIGS. 4A and 4B, the front layer 400 may include a plurality of openings 402 and/or a cavity 404. The openings 402 (e.g., cylindrical holes or cutouts) may be configured to allow a first subset of sensors (e.g., one or more EEG sensors, one or more EOG sensors, one or more EDA sensors, and/or one or more EMG sensors) of the sensing device to pass through the front layer and contact the user's face. The cavity 404 (e.g., an opening located at the center of the front layer) may be configured to expose a second subset of sensors (e.g., one or more PPG sensors) of the sensing device to the user's face and allow those sensors to collect signals (e.g., optical signals) from the user's face.

FIG. 4C shows that the front layer 400 may include multiple sublayers each having the openings 402 (e.g., cylindrical holes) to allow the sensors described herein to pass through and contact the user's face. The multiple sublayers may include, for example, a surface finish sublayer 400a configured to contact the skin of the user's face, an ethylene vinyl acetate (EVA) moldable foam sublayer 400b (e.g., located next to the surface finish sublayer) configured to be compressible to relieve pressure and/or accommodate different face shapes, a memory foam sublayer 400c (e.g., located next to the EVA moldable foam sublayer and further away from the user's face) configured to be compressible to provide further pressure relief and/or accommodate different face shapes, and/or an electromagnetic shielding sublayer 400d (e.g., located furthest away from the user's face and/or next to the PCB layer 304) to serve as an EMF shield.

With the sublayers 400a-400d, the front layer 400 may be able to distribute pressure exerted by the sensing device to the user's face across a larger area, thus reducing the PSI (pound per square inch) in a specific location. The sublayers may also operate to separate the electrical components of the sensing device and the user's face (e.g., preventing circuitry of the PCB layer 304 from directly contacting the user's face). Such separation may improve the user's comfort while also protecting the electrical components from erosion and/or wear and tear. In examples, the surface finish sublayer 400a of the front layer 400 may be made of a breathable material to further increase the comfort level of the user. Having such a surface finish sublayer may also make it easier to wipe/clean the front layer 400.

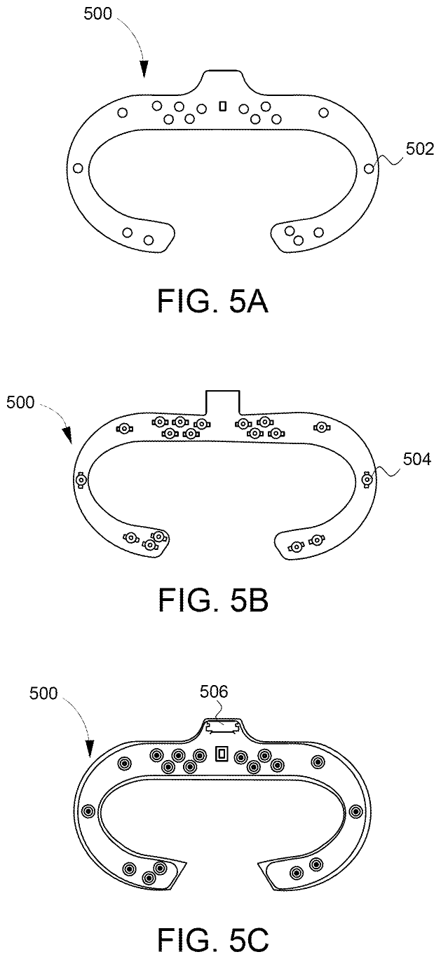

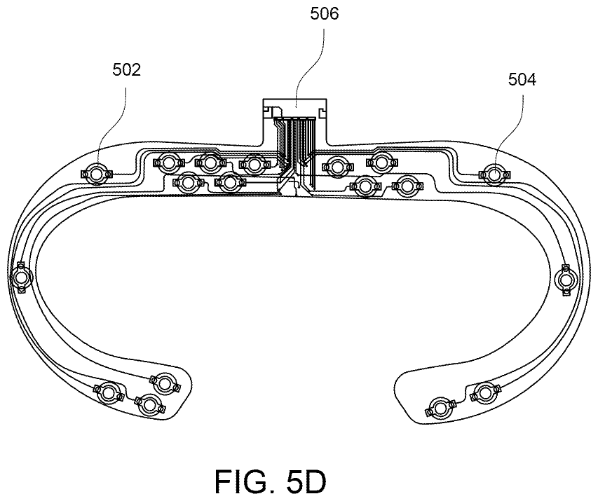

FIGS. 5A-5D show an example PCB layer 500 (e.g., the PCB layer 304 in FIGS. 3A and 3B) of the sensing device described herein. As shown, the PCB layer 500 may include one or more snap connectors 502 (e.g., 18 female snap connectors) configured to secure respective sensors 504 (e.g., 18 electrodes such as 304a shown in FIGS. 3A and 3B) to the PCB layer 500. The PCB layer 500 may further include a PCB 506 (e.g., the PCB 304b in FIGS. 3A and 3B) electrically coupled to one or more (e.g., all) of the sensors 504 and configured to process the biometric signals collected by the sensors 504. The PCB 506 may be further communicatively coupled to other circuits (e.g., circuits internal and/or external to the sensing device) and exchange information with these other circuits (e.g., transmit the signals collected by the sensors 504 and/or pre-processed by the PCB 506 to these other circuits).

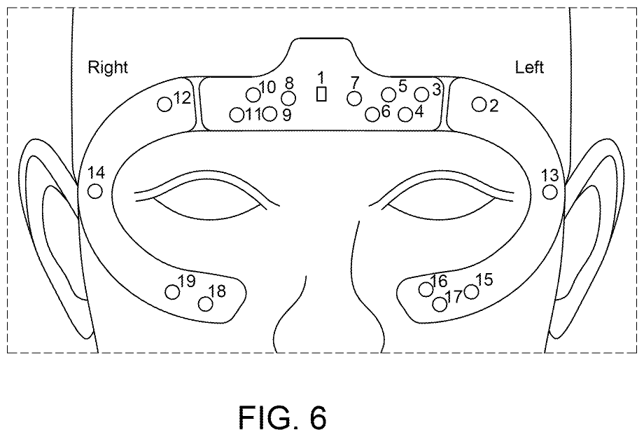

The snap connectors 502 and/or the sensors 504 may be placed at selected locations of the PCB layer 500 so that the sensors 504 may contact (e.g., through the openings 402 shown in FIGS. 4A and 4B) respective areas of the user's face to collect biometric signals from the user. For instance, the sensors 504 may be divided into groups for collecting EEG, EMG, EDA, and EOG signals (e.g., simultaneously or within a same signal collection session). FIG. 6 shows example placement of the sensors 504. For instance, two sensors (e.g., among those labeled 3-6, 9-11, or 13-19) may be used to sense and/or collect EEG signals, eight sensors (e.g., among those labeled 3-6, 9-11, or 13-19) may be used to sense and/or collect EMG signals, four sensors (e.g., among those labeled 3-6, 9-11, or 13-19) may be used to sense and/or collect EOG signals, and two sensors (e.g., labeled 7 and 8) may be used to sense and/or collect EDA signals. Further, one or more BIAS sensors (e.g., the sensor labeled 2) and/or one or more SRB2 sensors (e.g., the sensor labeled 12) may be included and used as reference points for evaluating the voltage measurements at other sensor locations. An example assignment of the sensors based on the labeling shown in FIG. 6 is shown in Table 1 below.

TABLE 1
Example Sensor Assignment

| Data Type | Sensor Locations |
|---|---|
| PPG | 1 |
| BIAS | 2 |
| SRB2 | 12 |
| EDA | 7, 8 |
| EEG | 5, 10 |
| EMG | 4, 6, 9, 11, |
| | 15, 16, 18, 19 |
| EOG | 3, 13, 14, 17 |

The assignment and/or operation of the sensors 504 (e.g., the assignment and/or operation of EEG, EMG, and EOG sensors) may be configurable, for example, by a control device (e.g., the server 104 and/or the computer device(s) 110 shown in FIG. 1) and/or using firmware embedded in the PCB 506. Different sensors may be designated to sense and/or collect one or more types of the biometric signals described herein. For instance, a sensor may be dynamically switched from collecting EEG signals to collecting EMG signals, or vice versa. Other sensor settings such as PGA gains and/or whether a Bias or SRB2 sensor location is to be used as a reference point to calculate the voltage sensed by a specific sensor may also be configurable.

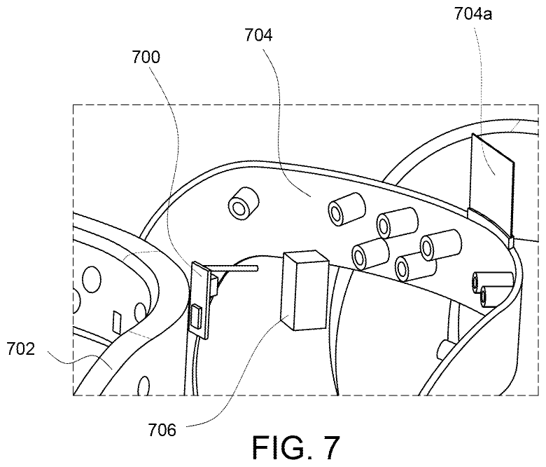

FIG. 7 shows an example of a PPG PCB 700 that may be secured between a front layer 702 (e.g., the front layer 302 in FIGS. 3A and 3B) of the sensing device described herein and a PCB layer 704 of the sensing device described herein (e.g., the PCB layer 304 shown in FIGS. 3A and 3B). The PPG PCB 700 may be attached to (e.g., supported by) a structure 706 that may be coupled to the PCB layer 704 (e.g., inside of the front layer 702). The PPG PCB 700 may include or may be coupled to a PPG sensor configured to pass through a cavity (e.g., opening) in the front layer 702 (e.g., the cavity 404 in FIGS. 4A and 4B) and detect signals (e.g., optical signals) from the user's face. Such signals may be based on blood volume changes in the microvascular bed of a facial tissue. For instance, the PPG sensor may include a photodiode capable of illuminating the user's facial skin (e.g., using a pulse oximeter such as an LED) and the PPG PCB 700 may be configured to determine PPG information about the user based on light absorption changes measured by the photodiode. The PPG PCB 700 may be electrically and/or communicatively coupled (e.g., via a communication cable) to circuitry 704a (e.g., the PCB 506 in FIGS. 5A-5D) of the PCB layer 704 and pass the PPG information to the circuitry 704a.

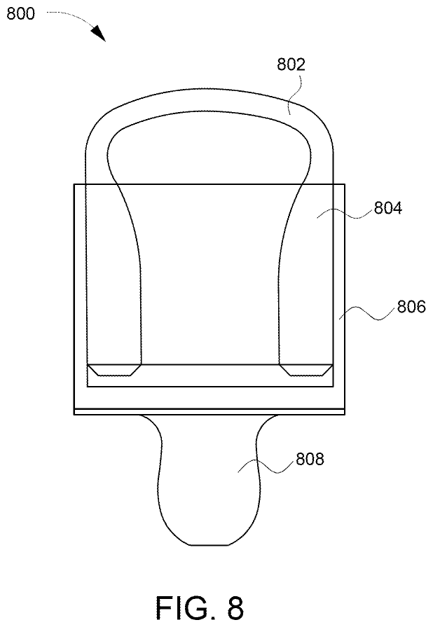

The sensors described herein (e.g., the sensors 502 of FIGS. 5A and 5B) may include a metal electrode (e.g., solid metal electrode) and/or a stylus electrode as described further below. The electrode may be capable of providing active amplification to a collected signal (e.g., the electrode may be an active electrode), or the electrode may be a passive electrode that does not provide amplification to the collected signal. FIG. 8 shows an example of a stylus electrode 800 that may be included in the sensors described herein for collecting a biometric signal from a user. As explained below, the electrode 800 may be comfortable, replaceable, and/or capable of maintaining close contact with a user's face (e.g., regardless of whether the user's head is stationary or moving).

The electrode 800 may include a conductive surface 802, a wall 804, a casing 806 (e.g., a cylindrical casing), and/or a snap connector 808 (e.g., a male snap connector). The conductive surface 802 may be made of a conductive polymer material. The wall 804 may also be made of a polymer material and, together with the conductive surface 802, may form a hollow center. The conductive surface 802 and/or the wall 804 may be enclosed within the casing 806, and at least a portion of the conductive surface 802 may extend beyond the top of the casing 806 to contact the user's face. The casing 806 may be made of a rigid conductive material and may form a part of a conductive path for the signals collected by the conductive surface 802. The bottom of the casing 806 may be connected to the snap connector 808 to form a base (e.g., the base may also be a part of the conductive path for the signals collected by the sensor 800), allowing the sensor 800 to be snapped into or out of a female connection point (e.g., the female snap connectors 502 shown in FIG. 5A). Having the ability to snap the sensor 800 in and out of the embedded flexible facepad PCB may render the sensor 800 replaceable and/or recyclable, thus reducing the costs associated with making, using, and/or maintaining the facepad described herein.

The conductive surface 802 may be made of silicone with conductive additive and/or EPDM rubber (ethylene propylene diene monomer rubber). Using these soft, flexible materials for the conductive surface 802 may result in the conductive surface being gentler and more comfortable to the user's face when pressure is applied (e.g., similar to the use of a stylus on a touch screen device). This may contrast with using a rigid metal material for the conductive surface 802, which may concentrate the force of connection on a smaller surface area, making the device less comfortable to the user's face. The hollow cavity surrounded by the conductive surface 802 and the wall 804 may encourage the conductive surface 802 to compress inwards toward the base of the sensor 800 when the device is in use. This way, a larger surface area of the conductive surface 802 may be in contact with the user's face, allowing for an increased flow of electrons into the sensor and improving the quality of signal collection.

The conductive surface 802 may be thinner than the wall 804 so that the conductive surface 802 may feel softer on the user's skin and may deform more easily under pressure. Further, making the conductive surface 802 thinner than the wall 804 may encourage the conductive surface 802 to bend more readily than other parts of the sensor 800 when pressure is applied. On the other hand, making the wall 804 thicker (e.g., and more rigid) may give the polymer insert more structure within the casing 806 and prevent the conductive surface 802 from flexing away from the wall 804 or the base of the sensor 800, thus securing the conductive path that may run between the user's face and the facepad PCB via the base of the sensor 800.

Although a stylus electrode is described with reference to FIG. 8, the sensor 800 is not limited to using such an electrode. For example, the sensor 800 may include an electrode comprising a contact surface that is made of a conductive material (e.g., a metal). Such an electrode (e.g., a solid metal electrode) may still be configured to be secured to the circuit board via the female snap connector described herein. For instance, part of the electrode (e.g., the base of the electrode) may be shaped as a male snap connector capable of being snapped into and out of the female snap connector, and the base of the electrode may form a part of a conductive path for the signal collected via the contact surface.

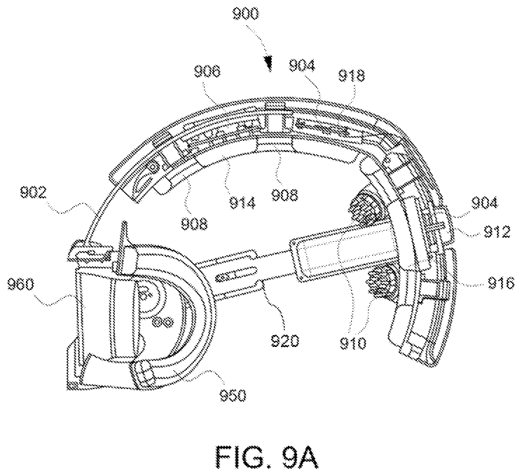

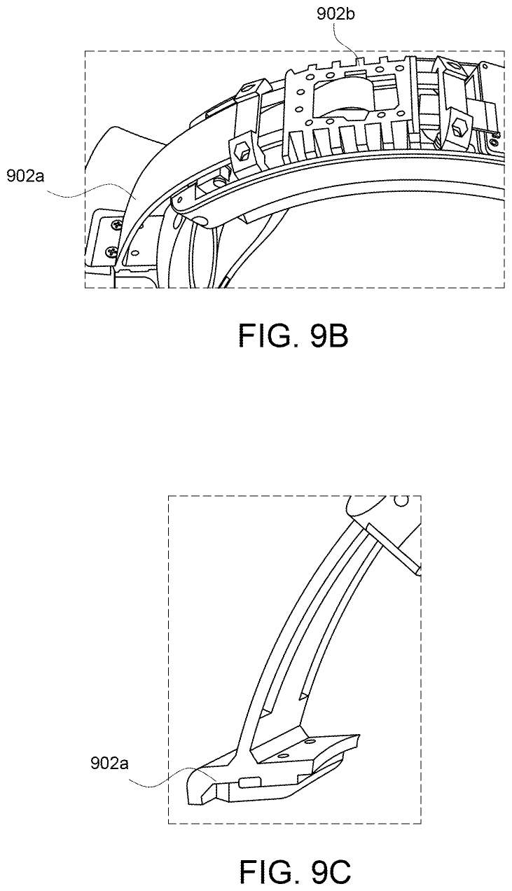

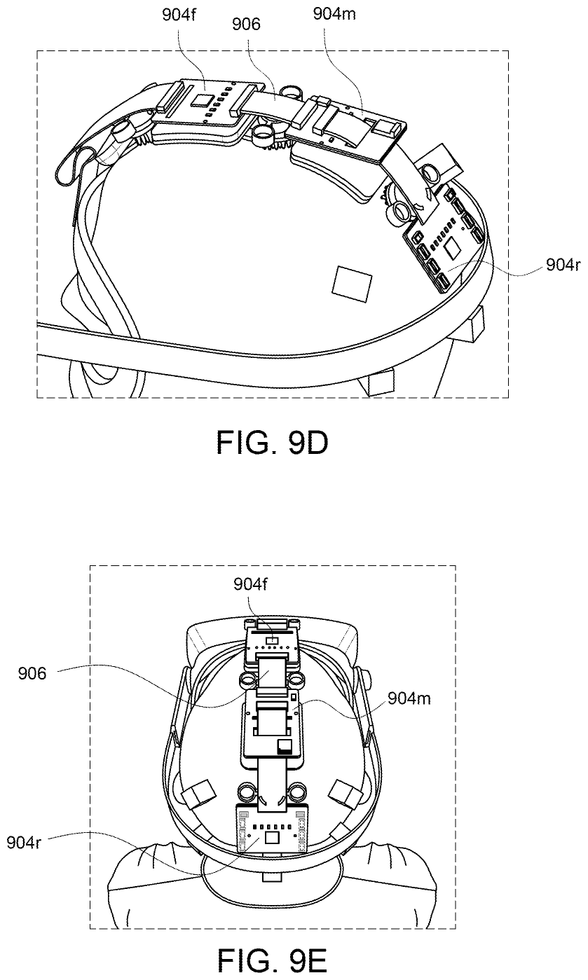

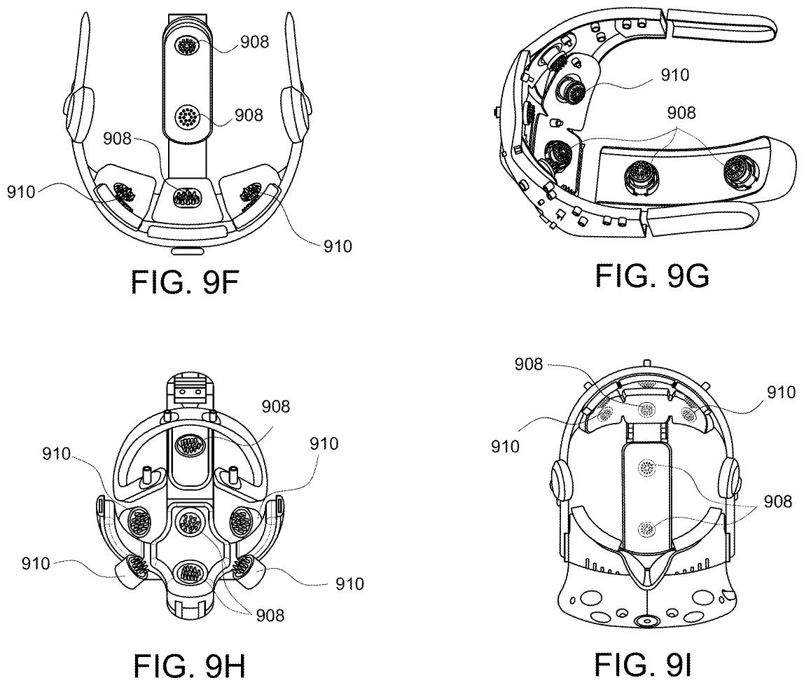

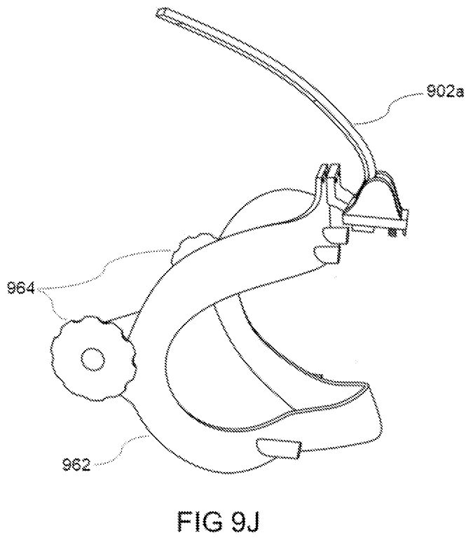

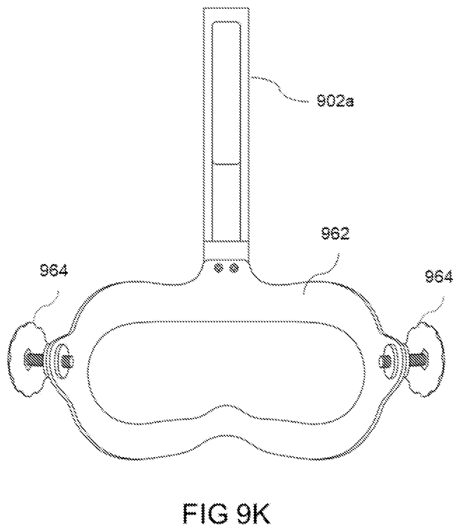

The sensing device described herein (e.g., the sensing device 202 of FIGS. 2A-2D or the sensing device 300 of FIGS. 3A and 3B) may be used in conjunction with other devices or apparatus (e.g., such as the device 204 shown in FIGS. 2A-2D) that may also be configured to collect biometric signals from a user. FIG. 9A illustrates a scalp engagement biometrics collection apparatus 900 (referred to herein as a “strapparatus”) that may be configured to be secured to a user's head and collect biometric signals from the user. The strapparatus 900 may be deployed standalone or it may be coupled to a sensing device 950 as described herein (e.g., the sensing device 202 of FIGS. 2A-2D or the sensing device 300 of FIGS. 3A and 3B) and/or an HMD 960, and exchange information with those devices. As shown, the strapparatus 900 may include a midline rail 902, one or more integrated circuits 904 (e.g., PCBs), one or more communication cables 906 (e.g., ribbon cables), one or more midline sensors 908 (e.g., midline EEG sensors or electrodes), one or more distributed sensors 910 (e.g., distributed EEG sensors or electrodes), a rear adjuster 912, a side arm 920, one or more pressure relief pads 914, a battery 916, and/or a communication circuit 918 (e.g., a WiFi transceiver). As will be described in greater detail below, the unique design of the strapparatus 900 may allow for not only accurate collection of the biometric signals but also increased comfort of the head mounted sensing device.

FIGS. 9B and 9C illustrate the midline rail 902 of the strapparatus 900. As shown, the midline rail 902 may include a guide arm 902a and/or a ribbon cable guide 902b through which the one or more communication cables 906 may run. The guide arm 902a may be configured to connect (e.g., mechanically and/or electronically) the strapparatus 900 to other devices such as the sensing device 950. The guide arm 902a may be extendable, for example, along at least a midline direction of the user's head, so that the strapparatus 900 may be adjusted to fit different head sizes and/or head shapes. The rear adjuster 912 may provide additional means for adjusting the strapparatus 900. For instance, the rear adjuster 912 may include a rotatable knob that may be turned to tighten or loosen the strapparatus to conform to the user's head size as well as to adjust the pressure applied to the user's scalp. In addition to rear adjuster 912, strapparatus 900 may include other types of adjustment mechanisms to ensure a tight fit of strapparatus 900 onto human heads of different sizes. FIG. 9J illustrates the interconnection between the guide arm 902a of the strapparatus 900 and a shell 962 that houses the sensing device 202 using an adjustment mechanism 964. Adjustment mechanism 964 may include one or more (e.g., two) threaded-screws that are inserted into a threaded hole on the sides of the shell 962. Each of the threaded-screws may include a knob that may be turned to tighten up or loosen the pressure between different layers, thus changing the internal curvatures of the four layers 302-308. FIG. 9K shows two adjustment mechanisms 964 each including a threaded screw with a knob that, when turned, may change the curvature of the four layers 302-308. As the knobs of the adjustment mechanisms 964 are turned, for example, by pushing and applying pressures against the side arms 920 of the strapparatus 900, a user can adjust the internal curvatures of the four layers 302-308 of the facepad, thereby improving the connection quality between the sensors on the sensing device 202 and the skin of the human body (e.g., the head).

The integrated circuits 904 may include one or more PCBs configured to be hosted on (e.g., attached to) the midline rail 902 (e.g., in respective PCB mounting receptacles). The PCBs may be electrically coupled to the sensors 908 and 910, and configured to process the biometric signals (e.g., EEG signals) collected by the sensors 908 and 910. The processing tasks may be carried out by one PCB or they may be divided among multiple PCBs communicatively coupled via the one or more communication cables 906. FIG. 9D and 9E show examples in which the strapparatus 900 may include a main PCB 904m (e.g., a main circuit board), a first physio PCB 904f (e.g., a front physio circuit board), and/or a second physio PCB 904r (e.g., a back physio circuit board) connected via the communication cables 906. These PCBs may include embedded electronics and/or programming logics configured to perform various signal processing tasks including, for example, converting the biometric signals detected by the sensors 908/910 from analog format to digital format (e.g., using one or more analog-to-digital converters (ADC)), preprocessing the biometric signals to remove noise and/or interference, tagging (e.g., associating) the biometric signals with corresponding timestamps, organizing the biometric signals according to user preferences, etc.

In examples, the main PCB 904m of the strapparatus 900 may include a processing unit (e.g., a CPU, a GPU, and/or a MPU) configured to provide a system clock for unifying (e.g., fusing, combining, and/or reconciling) the biometric signals collected by the various sensors described herein, e.g., to expand the application range of the derived biometric information. The main PCB 904m (and/or the first and second physio PCBs) may be communicatively coupled to other devices such as the sensing device 950 (e.g., the PCB 704a and/or PPG PCB 700 shown in FIG. 7) and fuse the signal/data streams collected by different types of sensors (e.g., EEG sensors, EOG sensors, EDA sensors, PPG sensors, and/or EMG sensors) into a time series (e.g., a single time series) so as to obtain a holistic view of the user's neural and/or physiological reactions to audio/visual stimuli. The main PCB 904m may also be configured to transmit the unified biometric information to a receiving device (e.g., the server 104 and/or computing device(s) 110 of FIG. 1) and/or receive control information from a control device (e.g., the server 104 and/or computing device(s) 110 of FIG. 1), for example, via the communication circuit 918. The division of functionality across multiple PCBs may provide flexibility to the strapparatus 900 while also allow the strapparatus to be closely aligned (e.g., since the PCBs may be made smaller) with the shape of the human head, thereby improving not only the sensitivity and accuracy of the signal collection but also the overall comfort level of the collection device.

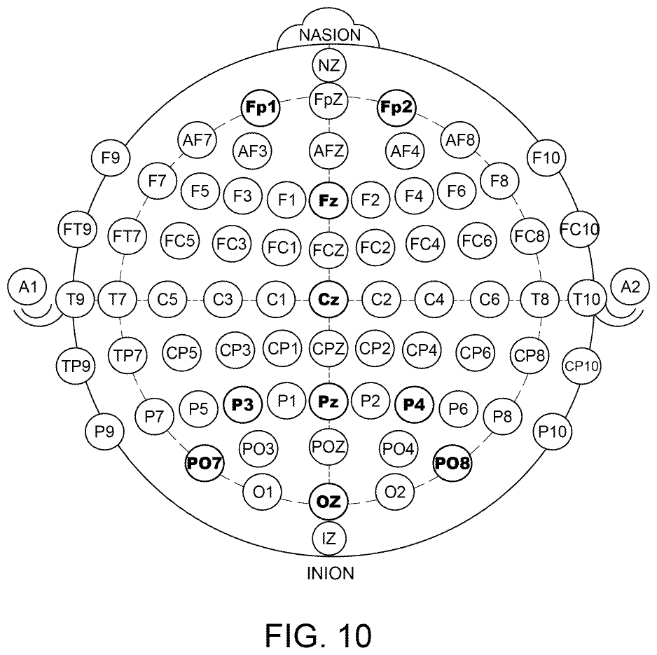

The midline sensors 908 and/or the distributed sensors 910 of the strapparatus 900 may each include an electrode (e.g., an active electrode) configured to collect biometric signals (e.g., EEG signals) from a respective area of the user's scalp. The midline electrodes may be positioned (e.g., in respective midline electrode receptacles) to align with the middle section of the user's scalp while the distributed electrodes may be positioned (e.g., in respective distributed electrode receptacles) to align with one or more occipital sections of the user's scalp, for example, as shown in FIGS. 9F-9I. FIG. 10 shows example locations of the midline and distributed electrodes in accordance with internationally recognized scalp electrode locations. As shown, one or more of the midline electrodes (e.g., 4 active electrodes) may be placed in the areas marked as Fz, Cz, Pz, and Oz, and one or more of the distributed electrodes (e.g., 4 active electrodes) may be placed in the occipital areas marked as P3, P4, PO7, and PO8.

The electrodes of the midline sensors 908 and/or distributed sensors 910 may be configured to maintain close contact with the user's scalp and be durable, replaceable, and comfortable to use. For instance, the electrodes may be implemented using flexible conductive materials that may deform in predictable manners when pressure is applied to the electrodes (e.g., once the strapparatus 900 is secured to the user's head). As another example, each electrode may include a plurality of conductive projections (e.g., combs, prongs, or spikes that may contact/engage the user's scalp) for collecting signals from multiple points of contact in and around the area where the electrode touches the user's scalp.

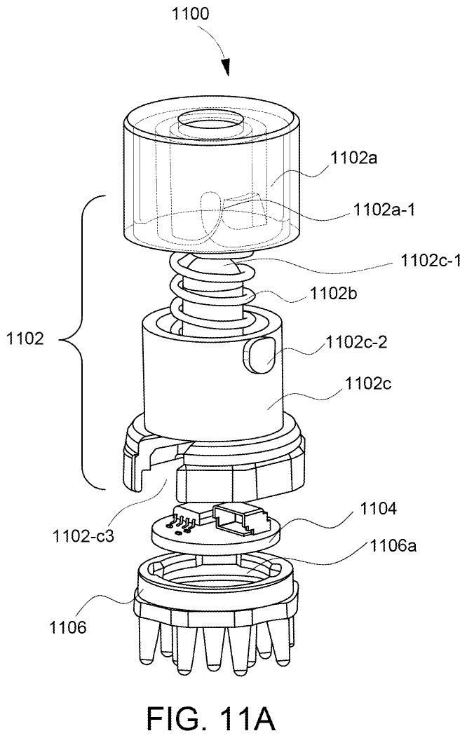

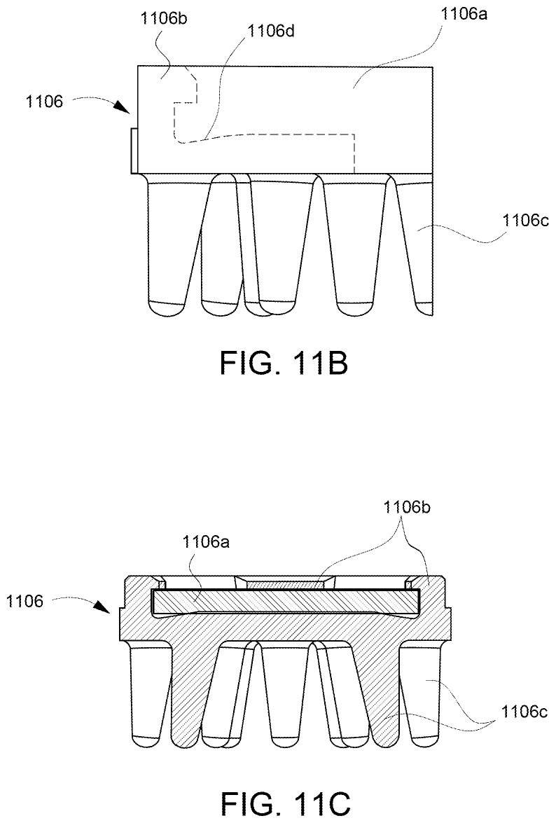

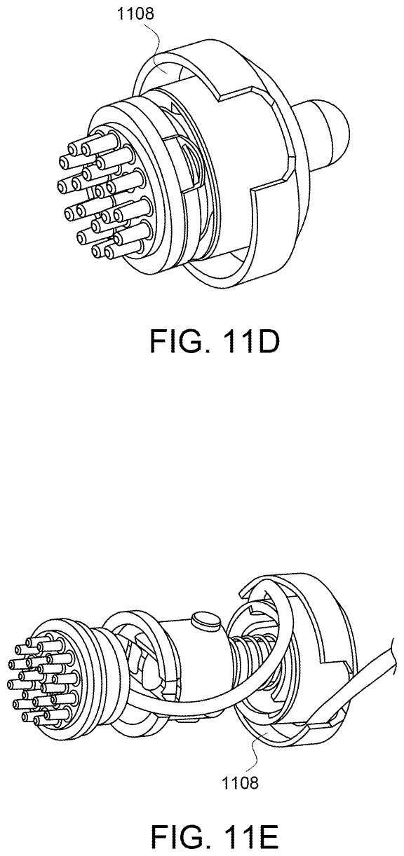

FIG. 11 illustrates an example electrode 1100 that may be included in the midline and/or distributed sensors described herein. As shown, the electrode 1100 may include a sabot assembly 1102, a circuit board 1104 (e.g., a PCB), and/or a conductive contact assembly 1106. Each of these components may be designed in manners that allow them to be combined modularly and/or replaced individually. The sabot assembly 1102 may include a cap 1102a, a spring 1102b, and a casing 1102c (e.g., a cylinder-shaped casing). Such a spring-loaded sabot assembly 1102 may operate to provide pressure relief to a user wearing the strapparatus described herein and/or ensure that the strapparatus be adaptable to account for differences in head size and head shape. For example, the cap 1102a may be configured to orient the sabot assembly 1102 towards the user's scalp and/or to provide a backstop for the spring 1102b. The cap 1102a may also serve as a connection point between the electrode 1100 and another device (e.g., an HMD) with which the electrode may be combined. The spring 1102b may provide pressure relief and ensure that the electrode 1100 maintain close contact with the user's scalp. The casing 1102c may include a peg 1102c-1 (e.g., a post), a protrusion 1102c-2, and/or a wiring channel 1102c-3. There may be a first opening at the bottom of the casing 1102c that allows the combination of PCB 1104 and conductive contact assembly 1106 to be fit into the bottom of the casing. There may also be a second opening along an outer wall of the casing 1102c that may serve as a channel for wiring between the PCB 1104 and a connected device. The peg 1102c-1 may be located at the center of the casing 1102c and be configured to hold the spring 1102b in place (e.g., the spring 1102b may be disposed around the peg 1102c-1). The peg 1102c-1 may extend through a hole at the top of the cap 1102a when the casing 1102c and the cap 1102a are locked together. The peg 1102c-1 may be manipulated to make fine adjustments to the position of one or more conductive prongs of the electrode 1100 while the electrode is in use, ensuring that the conductive prongs extend through the user's hair and maintain close contact with the user's scalp.

The casing 1102c and the cap 1102a may be configured so that the casing 1102c may be locked into place within the cap 1102a or unlocked from the cap 1102a, for example, by fully compressing the spring 1102b and twisting the casing 1102c into a locked or unlocked position. The protrusion 1102c-2 may be located on the outside of the casing's top edge and may be configured to fit into a locking track 1102a-1 of the cap 1102a, for example, along the inside of the cap's outer wall. This locking mechanism may allow for individual components to be easily replaced, while also preventing the casing 1102c, the PCB 1104, and the conductive contact assembly 1106 from becoming detached accidentally while in use. The locking mechanism may also allow the active electrode to be combined with (e.g., fit into) another device (e.g., a headset), for example, by inserting the cap 1102a into a receptacle included in or attached to the other device.

The PCB 1104 of the electrode 1100 may be configured to receive the signals (e.g., analog signals) collected via the conductive contact assembly 1106 and prepare the signals for further processing by other unit(s) or component(s) of the strapparatus. For example, the PCB 1104 may be configured to apply amplification (e.g., active amplification) to the analog electrical signals collected via the conductive contact assembly 1106 before passing the amplified signals to another unit or component for processing. While the examples may be described herein using active electrodes (e.g., capable of providing active amplification to the collected signals), part or all of the examples may also be implemented using other types of electrodes including, e.g., passive electrodes, which may not apply amplification to the collected signals.

The conductive contact assembly 1106 may be configured to enclose the PCB 1104, for example, in a press fit bed 1106a. The press fit bed 1106a may be made of a flexible and/or conductive material such as a conductive polymer, and be shaped and/or configured to maintain close contact with the PCB 1104. In examples, the press fit bed 1106a may have a raised (e.g., convex or curving outward) surface (e.g., a circular surface) at the bottom of the press fit bed that is configured (e.g., curved) to maximize the contact area between the press fit bed and the bottom surface (e.g., a metal bottom surface such as a copper surface) of the PCB 1104 when the PCB is pressed into the press bit bed. The surface of the press fit bed may flex predictably under pressure (e.g., as a characteristic of the polymer material from which the press fit bed may be made), securing the contact between the press fit bed 1106a and the PCB 1104 and increasing the number of electrons that may flow from the conductive contact assembly 1106 into the PCB 1104 when the two parts are assembled together.

FIGS. 11B and 11C show examples of the conductive contact assembly 1106 including the press fit bed 1106a described herein. The conductive contact assembly 1106 may also include one or more overhanging flanges 1106b located around the rim of the conductive contact assembly to hold a PCB (e.g., the PCB 1104 in FIG. 11A) in place, for example, by providing downward pressure on the top surface of the PCB. This pressure may cause one or more scalp engagement devices 1106c (e.g., the conductive prongs described below) to flex outward and result in the PCB being pressed into the center of the press fit bed 1106a. The center of the press fit bed 1106a may include a raised, flat, and/or circular surface and there may be a downward taper 1106d (e.g., at the outer edge closest to the walls of the conductive contact assembly) that is configured to give the press fit bed (e.g., which may be made of a polymer) room to flex as pressure is applied, without losing contact with the PCB (e.g., at the center of the bed). Maintaining secure contact between the PCB and the press fit bed 1106 may ensure that noise caused by movement of one or more the components described herein not be introduced into the signals acquired by the electrode.

FIGS. 11D and 11E show an example of the conductive contact assembly 1106 that includes a wire strain relief guide 1108.

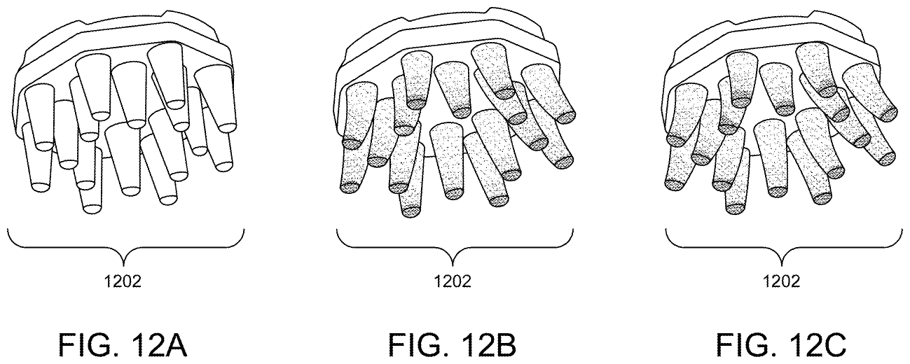

The conductive contact assembly 1106 described herein may include multiple (e.g., 16) scalp engagement devices or prongs 1106c (e.g., conical protrusions) that may be capable of extending through a user's hair and making contact with the user's scalp when the conductive contact assembly is pressed against the user's scalp. These prongs may be made of a conductive polymer and may be arranged to allow the prongs to predictably and comfortably bend outward under pressure to ensure signal detection as well as user comfort. FIGS. 12A-12C illustrate examples 1202 of the conductive prongs 1106c described herein. FIG. 12A shows the prongs 1202 under no pressure, FIG. 12B may show the example prongs 1202 under low pressure, and FIG. 12C may show the example prongs 1202 under high pressure. As shown in the examples, the prongs 1202 may be arranged into one or more concentric rings (e.g., two rings each comprising 8 prongs) around the center of the bottom of the conductive contact assembly (e.g., other non-ring type of arrangement such as arrays may also be used so long as the arrangement can accomplish the design goals described herein). In examples, the center (e.g., the absolute center) of the bottom surface itself may be left open (e.g., not occupied by any prongs) to increase the overall comfort of the active electrode and/or improve the contact between the electrode and a user's scalp (e.g., the open center may account for the natural curvature of a human scalp). Not having a central prong may prevent the majority of the force/pressure from being focused through the central prong and may allow the force/pressure to be dissipated into the surrounding radial prongs (e.g., 16 radial prongs). The outer octagonal shape of the conductive contact assembly shown in the examples may ensure safe and secure fitting of the conductive contact assembly into other components or devices, while also increase the aesthetic appeal of the assembly.

One or more (e.g., each) of the prong 1202 may be configured to angle away from the center of the conductive contact assembly such that the side furthest from the center may be perpendicular (e.g., substantially perpendicular) to the bed of the conductive contact assembly and the inner edge of the prong may be at an obtuse angle with the bed of the conductive contact assembly (e.g., the exact shape of a prong may be the same as or may be different from that of other prongs). Shaping and/or angling the prongs 1202 in these manners may encourage the prongs to bend outward relative to the center of the conductive contact assembly when pressure is applied, thus preventing the prongs from folding or bending in different directions that may reduce the quality of the signals collected via the prongs. The design and/or configuration of the prongs may also ensure that the prongs maintain uniform contact with a user's scalp and be comfort to the user's scalp. Further, the outward bending of the prongs may also enhance the contact between a press fit bed (e.g., the press fit bed 1106a of FIG. 11A) and a PCB (e.g., the PCB 1104 of FIG. 11A) described herein since the bed may bend as the prongs spread outwards, while maintaining close contact with the center of the PCB, where the signals collected from the user's scalp may be transmitted to the PCB. In examples, the conductive contact assembly and/or the prongs 1202 may be made of a flexible, conductive material such as silver powder in a silicone matrix, graphite in a 3D printed UV resin, and/or the like. In examples, the conductive contact assembly and/or the prongs 1202 may be treated with conductive coatings, such as Ag—AgCl, to further improve the quality of signal detection and/or collection (e.g., by reducing the electrical impedance between the prongs and the user's scalp).

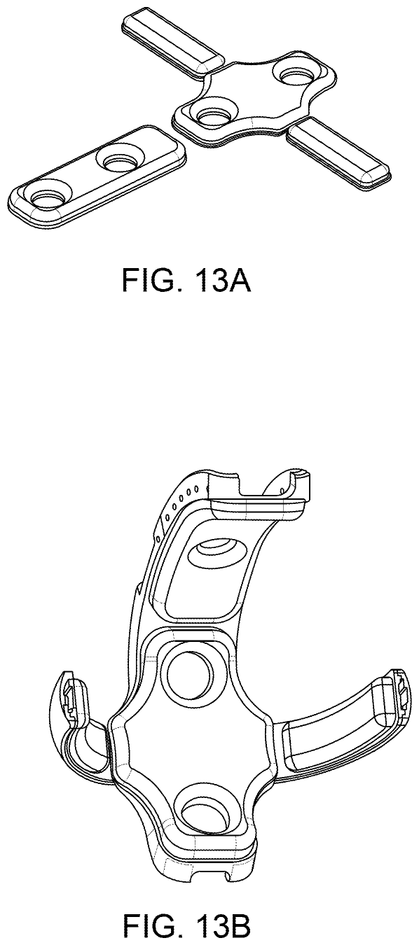

The strapparatus described herein may include one or more foam pads (e.g., memory foam pads) within which the sensors/electrodes described herein may be embedded. FIGS. 13A and 13B illustrate examples of these foam pads. As shown, the memory foam pads may include one or more openings (e.g., cutouts) into which the electrodes may be inserted. The foam pads may be made of materials that provide additional comfort to users of the strapparatus.

The systems and instrumentalities described herein may operate together with and/or be facilitated by machine-readable instructions (e.g., software and/or firmware) that may be stored in one or more memory devices and executable by one or more processors (e.g., CPUs, GPUs, MPUs, etc.). For example, when executed, these instructions (e.g., as a part of the firmware of the one or more PCBs described herein) may allow a user to initialize the systems or instrumentalities and/or to configure the settings of the systems or instrumentalities. The instructions may also cause the data collected by the systems or instrumentalities to be transmitted to a receiving device, for example, via a wired or wireless communication link (e.g., via a WiFi connection). The instructions may also allow users to initiate data collection sessions, troubleshoot and adjust sensor settings, visualize collected data alongside HMD content, integrate additional data streams, send data to other programs or services, etc. The data transmitted (e.g., to a receiving device or program) by the systems and instrumentalities described herein may be arranged in an array (e.g., a 2D array) comprising raw signal values in bytes. The receiving device or program may interpret the data array and render the data for visualizations relevant to the specific data type. For example, EEG data may be displayed as a timeseries, an FFT plot, a head plot, etc. When executed, the instructions described herein may also create one or more APIs for transmitting biometric data and/or metadata about the certain system and device configurations to a receiving API written in common programming languages such as Python, C++, C#, R, Java, MATLAB, and Julia.

A processing device as described herein may include a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. A communication circuit and/or communication link described herein may include a local area network (LAN), a wide area network (WAN), the Internet, a wireless data network (e.g., a Wi-Fi, 3G, 4G/LTE, or 5G network). A memory device described herein may include a storage medium configured to store machine-readable instructions that, when executed, cause a processing device to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. A memory device described herein may also include a mass storage device such as a magnetic disk (e.g., a hard drive), a removable disk, a magneto-optical disk, a CD-ROM or DVD-ROM disk, etc.

It should be noted even if some operations or functions are depicted and described herein with a specific order, these operations or functions may occur in various other orders, concurrently, and/or with other operations or functions not presented or described herein. Not all operations that the biosensing system is capable of performing are depicted and described herein, and not all illustrated operations are required to be performed by the biosensing system.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed:

1. A device configured to sense biometric signals from a user, comprising:
a multi-layered facepad, wherein the multi-layered facepad comprises:
a first layer comprising a plurality of openings and multiple sublayers;
a second layer comprising a circuit board, the circuit board comprising a plurality of sensors at least one of which is configured to pass through corresponding at least one of the plurality of openings to sense one or more of the biometric signals from the user;
a third layer comprising a compressible material configured to provide electromagnetic shielding for the second layer; and
a fourth layer configured to secure the first layer, the second layer, and the third layer to the device.

2. The device of claim 1, wherein the plurality of sensors is configured to sense the biometric signals from one or more areas of the user's face, the biometric signals indicating electroencephalography (EEG) information, electrooculography (EOG) information, electromyography (EMG) information, and electrodermal activity (EDA) information about the user.

3. The device of claim 2, further comprising a photoplethysmography (PPG) circuit board configured to obtain PPG information about the user.

4. The device of claim 3, wherein the PPG circuit board is configured to be attached to the circuit board of the second layer.

5. The device of claim 3, wherein the PPG circuit board comprises a plurality of optical sensors configured to sense optical signals that are indicative of the PPG information.

6. The device of claim 3, wherein the PPG circuit board is communicatively connected to the circuit board on the second layer and configured to transmit the PPG information about the user to the circuit board on the second layer.

7. The device of claim 1, wherein the second layer is configured to be sandwiched between the first layer and the third layer, and the third layer is configured to be sandwiched between the second layer and the fourth layer.

8. The device of claim 7, wherein the fourth layer comprises a gasket configured to hold the first, second, and third layers in place and physically connect the first, second, and third layers to the device.

9. The device of claim 7, wherein the circuit board is a flexible circuit board capable of deformation when pressure is applied to the circuit board or when the circuit board is bent or curved to fit the user's face.

10. The device of claim 1, wherein the multiple sublayers of the first layer include a surface finish sublayer configured to contact the user's face and an electromagnetic shielding sublayer configured to provide electromagnetic shielding for the circuit board.

11. The device of claim 10, wherein the multiple sublayers of the first layer further include an ethylene vinyl acetate (EVA) sublayer capable of being molded into different shapes and a memory foam sublayer.

12. The device of claim 1, wherein each of the plurality of sensors includes an electrode that comprises a contact surface made of a conductive material, the contact surface configured to contact the user's face when the device is secured to the user's face.

13. The device of claim 12, wherein the electrode is configured to be secured to the circuit board via a female connector.

14. The device of claim 13, wherein the conductive material comprises a metal.

15. The device of claim 13, wherein a part of the electrode is shaped as a male connector capable of being snapped into and out of the female snap connector.

16. The device of claim 12, wherein the electrode comprises a base that forms a part of a conductive path for a signal collected via the contact surface.

17. The device of claim 1, further comprising:
a scalp engagement device including a top side and a bottom side opposite the top side, the scalp engagement device further including a first printed circuit board (PCB) mounting receptacle on the top side, a plurality of electrode mounting receptacles on the bottom side, and an extendable midline rail coupled to and running between the top side and the bottom side of the scalp engagement device; and
a plurality of electrodes each configured to be removably hosted in a respective one of the electrode mounting receptacles of the scalp engagement device, the plurality of electrodes configured to contact the user's scalp and collecting one or more of the biometric signals from the user's scalp.

18. The device of claim 17, further comprising a first PCB configured to be hosted in the first PCB mounting receptacle of the scalp engagement device, the first PCB configured to process the biometric signals collected by the plurality of electrodes.

19. The device of claim 17, wherein each of the plurality of electrodes comprises a sabot assembly, an electrical circuit board, and a conductive contact assembly.

20. The device of claim 19, wherein the sabot assembly of each of the plurality of electrodes is configured to be removably coupled with the respective one of the electrode mounting receptacles.

21. The device of claim 20, wherein the sabot assembly comprises a spring configured to provide pressure relief to the user's scalp, a cap configured to operate as a backstop of the spring, and a casing coupled with the cap and configured to host the spring.

22. The device of claim 21, wherein the casing comprises a post configured to hold the spring in place, a protrusion configured to fit into a locking track of the cap, and a channel configured to allow wiring to pass from the electrical circuit board to outside the electrode.

23. The device of claim 19, wherein the conductive contact assembly of each of the plurality of electrodes comprises an array of flexible prongs arranged in a concentric pattern.

24. The device of claim 23, wherein each of the flexible prongs has a substantially oblique conical shape with an apex of the conical shape arranged radially away from a base of the conical shape.

25. The device of claim 24, wherein the flexible prongs are capable of extending through the user's hair and contacting the user's scalp.

26. The device of claim 25, wherein the flexible prongs are made of a conductive polymer.

27. The device of claim 24, wherein the flexible prongs are configured to flex outwardly from a center point to intersect the user's scalp when downward force is applied upon the electrode.

28. The device of claim 27, wherein the conductive contact assembly further comprises a substantially convex bed with a raised center that tapers off toward a perimeter of the bed and wherein application of downward force upon each of the plurality of electrodes as it engages the user's scalp causes the flexible prongs intersecting the user's scalp to flex radially outwardly from the center point and further causes the perimeter of the bed to flex toward an electrically conductive surface of the electrical circuit board.

29. The device of claim 19, wherein the electrode is modular allowing for one or more of the sabot assembly, the electrical circuit board, or the conductive contact assembly to be replaced.

30. The device of claim 17, further comprising at least one of a first adjuster configured to adjust a length of the midline rail.

31. The device of claim 1, further comprises a side arm and a facepad adjuster connected to the side arm, the facepad adjuster including a threaded screw having a knob that, when turned using the knob, changes a curvature of at least one of the first layer, the second layer, the third layer, or the fourth layer.

32. The device of claim 17, wherein the electrical circuit board is further configured to provide the pre-processed biometric signals to a receiving device, and wherein the pre-processing of the biometric signals comprises amplifying the biometric signals.

33. A method for collecting biometric information about a user, the method comprising:
collecting first signals from a first set of one or more areas of the user's face using a first plurality of sensors, wherein the first plurality of sensors is removably installed on a circuit board sandwiched between a first layer and a second layer of a multi-layered facepad, the first plurality of sensors is configured to contact the first set of one or more areas of the user's face through a first plurality of openings in the first layer, and the first signals indicate electroencephalography (EEG) information about the user;
collecting second signals from a second set of one or more areas of the user's face using a second plurality of sensors, wherein the second plurality of sensors is removably installed on the circuit board, the second plurality of sensors is configured to contact the second set of one or more areas of the user's face through a second plurality of openings in the first layer, and wherein the second signals indicate electrooculography (EOG) information about the user;
collecting third signals from a third set of one or more areas of the user's face using a third plurality of sensors, wherein the third plurality of sensors is removably installed on the circuit board, the third plurality of sensors is configured to contact the third set of one or more areas of the user's face through a third plurality of openings in the first layer, and wherein the third signals indicate electromyography (EMG) information about the user;
processing the first, second, and third signals to determine the biometric information about the user; and
transmitting the biometric information to a receiving device.

34. The method of claim 33, further comprising collecting fourth signals from a fourth set of one or more areas of the user's face using a fourth plurality of sensors and processing the fourth signals in addition to the first, second, and third signals to determine the biometric information about the user, wherein the fourth plurality of sensors is removably installed on the circuit board, the fourth plurality of sensors is configured to contact the fourth set of one or more areas of the user's face through a fourth plurality of openings in the first layer, and wherein the fourth signals are indicative of electrodermal activity (EDA) information about the user.

35. The method of claim 33, further comprising collecting fifth signals via a photoplethysmography (PPG) circuit board and processing the fifth signals in addition to the first, second, and third signals to determine the biometric information about the user, wherein the fifth signals indicate PPG information about the user.

36. The method of claim 35, wherein the fifth signals include optical signals and the PPG circuit board includes a plurality of optical sensors configured to collect the optical signals.

37. The method of claim 35, wherein the PPG circuit board is communicatively connected to the circuit board sandwiched between the first layer and the second layer.

38. The method of claim 33, wherein the first plurality of sensors, the second plurality of sensors, and the third plurality of sensors each comprises an electrode.

39. The method of claim 38, wherein the electrode comprises a contact surface made of a conductive material, the contact surface configured to contact the user's face when the multi-layered facepad is secured to the user's face.

40. The method of claim 33, wherein processing the first, second, and third signals to determine the biometric information about the user comprises reconciling respective timestamps associated with the first, second, and third signals based on a system clock.

41. The method of claim 33, wherein the receiving device includes a computer, and wherein the transmission of the biometric information is performed via a wireless communication link to the computer.
